(19) United States
(12) Patent Application Publication
(10) Pub. No.: US 2003/0034248 A1
(43) Pub. Date: Feb. 20, 2003

(54) Electrodeposition baths containing boron-containing compounds and methods related thereto

(76) Inventor: Alan J. Kaylo, Glenshaw, PA (US)

Correspondence Address: PPG Industries, INC, Intellectual Property Department, One PPG Place, Pittsburgh, PA, 15272, US

(21) Appl. No.: 09/919,093

(22) Filed: Jul. 31, 2001

(51) Int. Cl. B32B 9/04; C25D 1/12; C07K 1/26

(52) U.S. Cl. 204/471; 204/502; 204/505; 204/506; 428/411.1

(57) ABSTRACT

Disclosed is an improved electrodeposition bath having a reduced volatile organic content. The bath contains a resinous phase dispersed in an aqueous medium, the resinous phase including an active hydrogen-containing ionic electrodepositable resin and a curing agent therefor. The improved electrodeposition bath further includes a boron-containing compound in an amount sufficient to retard the growth of microorganisms in the bath. Also disclosed is a method of electrocoating a conductive substrate using the improved electrodeposition bath of the invention. Substrates which are coated using the method of the invention are also disclosed.

### FIELD OF THE INVENTION

The present invention relates to an improved electrodeposition bath containing a resinous phase dispersed in an aqueous medium, the resinous phase comprised of an ionic electrodepositable resin, a curing agent therefor; and a boron-containing compound; and to the use thereof in the method of electrodeposition.

### BACKGROUND OF THE INVENTION

Electrodeposition as a coating method has become increasingly important in the coatings industry. Initially, electrodeposition was conducted with the workpiece being coated serving as the anode. This was familiarly referred to as anionic electrodeposition. However, in 1972, cationic electrodeposition was introduced commercially. Since that time, cationic electrodeposition has steadily gained in popularity and today is by far the most prevalent method of electrodeposition. Throughout the world, more than 80 percent of all motor vehicles produced are given a primer coating by cationic electrodeposition.

As compared with non-electrophoretic coating means, electrodeposition offers increased paint utilization, improved corrosion protection and relatively low environmental contamination. Electrodeposition typically offers environmental advantages because electrodepositable coating compositions contain very little organic solvent, and downstream processes such as closed loop rinsing, discussed below, minimizes loss of coating components to the surrounding environment during coating application.

The electrodeposition process involves immersing an electroconductive substrate into a bath of an aqueous electrocoating composition, the substrate serving as a charged electrode in an electrical circuit comprising the electrode and an oppositely charged counter-electrode. For example, in the case of a cationic electrocoat composition, the workpiece serves as a cathode. Sufficient electrical current is applied between the electrodes to deposit a substantially continuous, adherent film of the electrocoating composition onto the surface of the electroconductive substrate. The electrocoated substrate is then conveyed to a rinsing operation where it is rinsed with an aqueous rinsing composition.

Typical rinsing operations have multiple stages which can include closed loop spray and/or dip applications. For example, in a spray rinse application the electrocoated substrate exits the electrocoating tank and is conveyed over a rinse tank while an aqueous rinsing composition is spray applied to the electrocoated surfaces of the substrate. Excess rinsing composition is permitted to drain from the substrate into the rinse tank below. The rinsing composition is then recirculated to the spraying apparatus for subsequent spray applications. In a typical electrocoat operation, the electrodeposition bath is ultrafiltered to remove ionic contaminants and the =ultrafiltrate” is used in the rinsing operations.

Recirculating the coating or rinsing compositions is both economically and environmentally desirable. However, the combination of organic nutrients, for example organic neutralizing agents such as lactic acid, warmth, aeration and recirculation in an aqueous coating system can create an environment conducive to the growth of microorganisms such as algae, fungi and bacteria. These microorganisms, if left unchecked, can adversely affect the quality and appearance of the electrodeposited coating. Further, the presence of microorganisms in the electrocoating or rinsing composition can cause the formation of precipitates in the tanks, and variation in process parameters, for example, pH, conductivity, film build, throwpower and stability. Also, particulate “dirt” deposition and biofouling can occur, thereby detrimentally affecting the appearance of the applied coating and reduce system performance.

In early electrodeposition processes, the “ultrafiltrate” used in the rinse stages typically contained solvents, heavy metals, and other organic materials which assisted in the suppression of the aforementioned microorganism growth. However, in recent years, manufacturers of organic coatings, including electrodeposition coating compositions, have come under increasing pressure to reduce environmentally undesirable components such as volatile organic compounds (VOC), hazardous air pollutants (HAPs), and heavy metals, such as lead and chrome. Ironically, however, recent reduction of VOC, HAPs and heavy metals in electrodepositable compositions has given rise to increased bacterial infestation, particularly in the electrocoat rinse stages.

A number of compounds for controlling the growth of bacteria in heavy metal-free, low organic solvent content-electrodeposition baths are known and have been used with varying degrees of success. Among these are known microbiocides such as silver ion, and oxidizing agents such as hydrogen peroxide and calcium hypochlorite. However, although effective for controlling the growth of microorganisms, silver ion is costly and can contribute to dirt formation the electrodeposition bath. Oxidizing agents such as hydrogen peroxide and calcium hypochlorite are extremely effective as a microbiocide, but can oxidize organic components of the electrodepositable composition if used too frequently or in large amounts.

A microbiocide composition containing a mixture of 5-chloro-2-methyl-4-isothiazolin-3-one and 2-methyl-4-isothiazolin-3-one (commercially available as KAYTHON® LX from Rohm and Haas Co.) has been used commercially in electrodeposition coatings and rinse compositions as the sole microorganism control composition. Although effective for inhibiting and/or controlling the growth of microorganisms in such systems, this microbiocide is relatively expensive and can cause a rougher appearance than a coating composition without this microbiocide. Moreover, such microbiocide compositions can contain, as inert ingredients, metal salts, for example, magnesium nitrate and magnesium chloride. The presence of metal ions of these salts in electrodeposition systems is undesirable because such metals can cause coating defects due to gas generation at the cathode. Furthermore, such a microbiocide typically is not included as a component in the coating composition, but, rather, is added to the electrodeposition system in an assembly plant setting. Microbiocides can lose their effectiveness over time as they are depleted from the bath and constant replenishment is necessary. Moreover, some of the microbiocides discussed above can require special handling and disposal means.

Halonitroalkanes, for example, 2-bromo-2-niitropropane-1,3-diol which is commercially available as CANGUARD® 409 from Angus Chemical Company are known for use in industrial water systems such as cooling water systems, paper and pulp mill systems, pools and electrodeposition baths, to control the growth of microorganisms. Although less costly than the mixture of 5-chloro-2-methyl-4-isothiazolin-3-one and 2-methyl-4-isothiazolin-3-one discussed above, this material can negatively affect the appearance of the applied coating if used at relatively high levels in electrodeposition baths. Further, such material can contribute to the build-up of bromide ion in the bath which can corrode of metallic parts, such as pipes and connectors, used in electrodeposition tank construction.

U.S. Pat. No. 4,732,905 discloses a biocidal composition comprising a synergistic admixture of 5-chloro-2-methyl-4-isothiazolin-3-one, 2-methyl-4-isothiazolin-3-one and 2-bromo-2-niitropropane-1,3-diol. The reference discloses the use of this composition to control microorganism growth in water systems.

U.S. Pat. No. 6,017,431 discloses sulfamic acid, an inorganic acid as a neutralizing agent for cationic electrocoating compositions and for the adjustment of pH of the electrodeposition bath compositions containing these compositions. Such electrodeposition baths are more resistant to the adverse effects of microorganism growth when the amount of sulfamic acid in the electrodepositable composition is greater than 90 up to 100 equivalent weight percent. However, due to certain processing issues which can arise during the preparation of electrodeposition composition components containing sulfamic acid as the neutralizing agent, the inclusion of an organic acid in such electrodepositable compositions often is desirable. As mentioned above, however, organic acids, which are present to rectify these difficulties, can be consumed by bacteria. Moreover, in such cases, the indigestible sulfamic acid can be post-added to the electrodepositable composition to replace the organic acids consumed by bacteria.

U.S. Pat. Nos. 3,937,679; 3,959,106; 3,975,346; 4,001,101 disclose the use of boric acid as a solubilizing agent for ionic group-containing film-forming resins having onium salt groups, such as quaternary ammonium groups and ternary sulfonium groups. These resins are useful as a component in electrodepositable compositions, particularly cationic compositions. The use of boric acid as a solubilizing or neutralizing agent for a cationic resin comprising a commercially useful cationic electrodepositable composition would imply to one skilled in the art that the boric acid is present in the bath in an amount sufficient to influence critical operating parameters of an electrodeposition bath, such as pH, conductivity, throwpower and the like.

U.S. Pat. No. 4,443,569 discloses cathodically electrodepositable compositions based on a nitrogen base-containing binder containing tertiary amino groups and primary and/or secondary hydroxyl groups, and a metal compound. The metal compounds include the octoates, naphthenates, borates and acetyl acetonates of metals such as cobalt, copper, lead, nickel, and/or manganese which are required to be sparingly soluble or insoluble in water. In other words, these metal-containing compounds provide electrocoating compositions contain significantly less metal ions, or no metal ions at all dissolved in the aqueous phase. It is disclosed that these metal-containing electrodepositable compositions provide improvements in adhesion of the cathodically applied coatings to non-phosphatized steel substrates which may have residual drawing oils at the substrate surface.

JP 07331130A discloses a cationic electrodeposition coating composition comprising (A) zinc borate, (B) an amine-modified epoxy resin, and (C) a blocked polyisocyanate curing agent. This composition is tin-free and can be cured at a lower cure temperature that analogous compositions containing no zinc borate. Zinc borate has low or no solubility in the aqueous phase. Likewise, JP 06340831 discloses a method for coating steel and aluminum substrates with a cationic electrocoating composition comprising at least one of silicates, borates, chromates, molybdates and tungstenates of alkaline earth metals and zinc. Such compositions provide coatings, for example, automotive coatings, having excellent corrosion resistance, especially filiform corrosion resistance.

A “commercially useful electrodeposition bath” is one which comprise electrodepositable compositions containing film-forming resins having ionic salt groups, for example, epoxy-based resins having amine salt groups and/or sulfonium salt groups, wherein the pH is 7 or less. Cationic compositions having a pH greater than 7 typically are not commercially viable because of the inability to control or maintain the bath at this pH. That is, at pH greater than 7, such cationic compositions tend to adsorb carbon dioxide from the surrounding atmosphere and, consequently, drift below pH 7 overtime.

In view of the foregoing, a need exists for a heavy metal-free, low or no VOC electrodeposition bath which is inherently biodegradation resistant while maintaining excellent coating application conditions, coating appearance and performance properties. The elimination of the necessity to handle toxic microbiocides that often are used in electrodeposition baths neutralized with organic acids is also desirable. It was surprising to find that the use of an effective amount of boric acid or its equivalent in such heavy metal-free, low or no VOC-electrodeposition baths can decrease or eliminate altogether the need for the addition of microbiocides without the attendant difficulties of the aforementioned compositions and without influencing critical electrodeposition process parameters such as pH and conductivity of the bath. Levels of boric acid, or its equivalents, sufficient to render the electrodeposition bath biodegradation resistant have little or no effect on these parameters.

### SUMMARY OF THE INVENTION

In accordance with the present invention, an improved aqueous electrodeposition bath having a reduced volatile organic content is provided. The electrodeposition bath comprises a resinous phase dispersed in an aqueous medium. The resinous phase comprises (a) an active hydrogen group-containing electrodepositable resin having ionic salt groups; and (b) a curing agent having functional groups reactive with the active hydrogen-containing groups of the resin (a). The improvement comprises the inclusion of a boron-containing compound selected from at least one of boric acid, boric acid equivalents, and mixtures thereof in the electrodeposition bath in an amount sufficient to retard the growth of microorganisms in the electrodeposition bath.

In a particular embodiment, the invention resides in an improved electrodeposition bath having a reduced volatile organic content. The electrodeposition bath comprises an aqueous electrocoating composition comprising a resinous phase dispersed in an aqueous medium. The resinous phase comprises (a) an active hydrogen group-containing electrodepositable resin having cationic salt groups; and (b) a blocked polyisocyanate curing agent having functional groups reactive with the active hydrogen-containing groups of the resin (a). The improvement comprises the inclusion of a boron-containing compound selected from at least one of boric acid, boric acid equivalents, and mixtures thereof in the electrodeposition bath, wherein the boron-containing compound is present in an amount sufficient to retard the growth of microorganisms in the electrodeposition bath, but present in an amount insufficient to form the cationic salt groups.

Also provided is a method of electrocoating a conductive substrate serving as a charged electrode in an electrical circuit comprising the electrode and an oppositely charged counter electrode which are immersed in an aqueous electrodeposition bath described above, and substrates coated by the method.

### DETAILED DESCRIPTION OF THE INVENTION

Other than in the operating examples, or where otherwise indicated, all numbers expressing quantities of ingredients, reaction conditions and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical values, however, inherently contain certain errors necessarily resulting from the standard deviation found in their respective testing measurements.

Also, it should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10.

Generally, the electrodeposition bath of the present invention has a reduced volatile organic content and comprises a resinous phase dispersed in an aqueous medium. The resinous phase comprises (a) an active hydrogen group-containing ionic electrodepositable resin, and (b) a curing agent having functional groups reactive with the active hydrogen groups of (a). As aforementioned, the improvement comprises the inclusion in the electrodeposition bath of a boron-containing compound selected from boric acid, boric acid equivalents, and mixtures thereof in an amount sufficient to retard the growth of microorganisms.

As used herein in the specification and in the claims, by “reduced volatile organic content” is meant an electrodeposition bath having a volatile organic content of 1.0 or less pounds per gallon, often 0.5 pounds per gallon, and typically 0.3 or less.

Suitable boron-containing compounds include those selected from boric acid, boric acid equivalents, and mixtures thereof. As used herein and in the claims, by “boric acid equivalents” is meant any of the numerous boron-containing compounds which can hydrolyze in aqueous media to form boric acid. Specific, but non-limiting examples of boric acid equivalents include boron oxides, for example, B₂O₃; boric acid esters such as those obtained by the reaction of boric acid with an alcohol or phenol, for example, trimethyl borate, triethyl borate, tri-n-propyl borate, tri-n-butyl borate, triphenyl borate, triisopropyl borate, tri-t-amyl borate, tri-2-cyclohexylcyclohexyl borate, triethanolamine borate, triisopropylamine borate, and triisopropanolamine borate, Additionally, amino-containing borates and tertiary amine salts of boric acid may be useful. Such boron-containing compounds include, but are not limited to, 2-(beta-dimethylaminoisopropoxy)-4,5-dimethyl-1,3,2-dioxaborolane, 2-(beta-diethylaminoethoxy)-4,4,6-trimethyl-1,3,2-dioxaborinane, 2-(beta-dimethylaminoethoxy)-4,4,6-trimethyl-1,3,2-dioxaborinane, 2-(betha-diisopropylaminoethoxy-1,3,2-dioxaborinane, 2-(beta-dibutylaminoethoxy)-4-m46hyl-1,3,2-dioxaborinane, 2-(gamma-dimethylaminopropoxy)-1,3,6,9-tetrapxa-2-boracycloundecane, and 2-(beta-dimethylaminoethoxy)-4,4-(4-hydorxybutyl)-1,3,2-dioxaborolane. Boric acid equivalents can also include metal salts of boric acid (i.e., metal borates) provided that such metal borates can readily dissociate in aqueous media to form boric acid. Suitable examples of metal borates useful in the electrodeposition bath of the present invention include, for example, calcium borate, potassium borates such as potassium metaborate, potassium tetraborate, potassium pentaborate, potassium hexaborate, and potassium octaborate, sodium borates such as sodium metaborate, sodium diborate, sodium tetraborate, sodium pentaborate, sodium perborate, sodium hexaborate, and sodium octaborate, Likewise, ammonium borates can be useful. Moreover, optional boron-containing compounds can be included, for example, bismuth borate and yttrium borate.

Suitable boric acid equivalents can also include organic oligomeric and polymeric compounds comprising boron-containing moieties. Suitable examples include polymeric borate esters, such as those formed by reacting an active hydrogen-containing polymer, for example, a hydroxyl functional group-containing acrylic polymer or polysiloxane polymer, with boric acid and/or a borate ester to form a polymer having borate ester groups.

Polymers suitable for this purpose can include any of a variety of active hydrogen-containing polymers such as those selected from at least one of acrylic polymers, polyepoxide polymers, polyester polymers, polyurethane polymers, polyether polymers and silicon-based polymers. By “silicon-based polymers” is meant a polymer comprising one or more —SiO— units in the backbone. Such silicon-based polymers can include hybrid polymers, such as those comprising organic polymeric blocks with one or more —SiO— units in the backbone. Boric acid is commonly used in the electrodeposition bath of the present invention.

In the electrodeposition bath of the present invention, the above-described boron-containing compound is present in the electrodeposition bath in an amount sufficient to provide an amount of boron of at least 50 parts per million, usually at least 200 parts per million, often at least 300 parts per million, and typically at least 500 parts per million, based on total weight of the electrodeposition bath. Also, the boron-containing compound is present in the electrodeposition bath in an amount sufficient to provide an amount of boron of less than 5000 parts per million, usually less than 4000 parts per million, often less than 3000 parts per million, and typically less than 2000 parts per million, based on total weight of the electrodeposition bath. The amount of boron present in the electrodeposition bath of the present invention can range between any combination of these values, inclusive of the recited values, so long as the amount is sufficient to provide a biodegradation resistant electrodeposition bath. As used herein, by “biodegradation resistant electrodeposition bath” is meant a bath which retards or is resistant to the growth of microorganisms such as bacteria, algae, fungi and the like, which can cause system and coating deficiencies such as those discussed above.

Moreover, the amount of boron-containing compound present in the electrodeposition bath of the present invention should be such that the electrodeposition is commercially useful as discussed above. That is, the boron-containing compound is present in the bath in an amount insufficient to influence critical operating parameters of the electrodeposition bath, such as pH, conductivity, throwpower, and stability. As previously discussed, the pH of the electrodeposition bath of the present invention is 7.0 or less, and typically can range from 5.2 to 7.0. Also the conductivity of the electrodeposition bath typically ranges from 500 to 3000 microsiemens as measured on a Model 50 pH/ion conductivity meter commercially available from Accumet.

In addition to the aforementioned boron-containing compounds, the electrodeposition baths of the present invention also contain, as a main film-forming polymer, an ungelled, active hydrogen-containing ionic, preferably cationic, electrodepositable resin. A wide variety of electrodepositable film-forming polymers are known and can be used in the electrodeposition baths of the invention so long as the polymers are “water dispersible,” i.e., adapted to be solubilized, dispersed or emulsified in water. The water dispersible polymer is ionic in nature, that is, the polymer will contain anionic functional groups to impart a negative charge or, as is preferred, cationic functional groups to impart a positive charge.

By “ungelled” is meant the resins are substantially free of crosslinking and have an intrinsic viscosity when dissolved in a suitable solvent, as determined, for example, in accordance with ASTM-D1795 or ASTM-D4243. The intrinsic viscosity of the reaction product is an indication of its molecular weight. A gelled reaction product, on the other hand, since it is of essentially infinitely high molecular weight, will have an intrinsic viscosity too high to measure. As used herein, a reaction product that is “substantially free of crosslinking” refers to a reaction product that has a weight average molecular weight (Mw), as determined by gel permeation chromatography, of less than 1,000,000.

Also, as used herein, the term “polymer” is meant to refer to oligomers and both homopolymers and copolymers. Unless stated otherwise, as used in the specification and the claims, molecular weights are number average molecular weights for polymeric materials indicated as “Mn” and obtained by gel permeation chromatography using a polystyrene standard in an art-recognized manner.

Examples of film-forming resins suitable for use in anionic electrodeposition bath compositions are base-solubilized, carboxylic acid containing polymers such as the reaction product or adduct of a drying oil or semi-drying fatty acid ester with a dicarboxylic acid or anhydride; and the reaction product of a fatty acid ester, unsaturated acid or anhydride and any additional unsaturated modifying materials which are further reacted with polyol. Also suitable are the at least partially neutralized interpolymers of hydroxy-alkyl esters of unsaturated carboxylic acids, unsaturated carboxylic acid and at least one other ethylenically unsaturated monomer. Still another suitable electrodepositable resin comprises an alkyd-aminoplast vehicle, i.e., a vehicle containing an alkyd resin and an amine-aldehyde resin. Yet another anionic electrodepositable resin composition comprises mixed esters of a resinous polyol. These compositions are described in detail in U.S. Pat. No. 3,749,657 at col. 9, lines 1 to 75 and col. 10, lines 1 to 13, all of which are herein incorporated by reference. Other acid functional polymers can also be used such as phosphatized polyepoxide or phosphatized acrylic polymers as are well known to those skilled in the art. Additionally, suitable for use as film-forming resins are those comprising one or more pendent carbamate functional groups, for example, those described in U.S. Pat. No. 6,165,338.

In one particular embodiment of the present invention, the active hydrogen-containing ionic electrodepositable resin (a) is cationic and capable of deposition on a cathode. Examples of such cationic film-forming resins include amine salt group-containing resins such as the acid-solubilized reaction products of polyepoxides and primary or secondary amines such as those described in U.S. Pat. Nos. 3,663,389; 3,984,299; 3,947,338; and 3,947,339. Usually, these amine salt group-containing resins are used in combination with a blocked isocyanate curing agent. The isocyanate can be fully blocked as described in the aforementioned U.S. Pat. No. 3,984,299 or the isocyanate can be partially blocked and reacted with the resin backbone such as described in U.S. Pat. No. 3,947,338. Also, one-component compositions as described in U.S. Pat. No.4,134,866 and DE-OS No. 2,707,405 can be used as the film-forming resin. Besides the epoxy-amine reaction products, film-forming resins can also be selected from cationic acrylic resins such as those described in U.S. Pat. Nos. 3,455,806 and 3,928,157.

Besides amine salt group-containing resins, quaternary ammonium salt group-containing resins can also be employed. Examples of these resins are those which are formed from reacting an organic polyepoxide with a tertiary amine salt. Such resins are described in U.S. Pat. Nos. 3,962,165; 3,975,346; and 4,001,101. Examples of other cationic resins are ternary sulfonium salt group-containing resins and quaternary phosphonium salt-group containing resins such as those described in U.S. Pat. Nos. 3,793,278 and 3,984,922, respectively. Also, film-forming resins which cure via transesterification such as described in European Application No. 12463 can be used. Further, cationic compositions prepared from Mannich bases such as described in U.S. Pat. No. 4,134,932 can be used.

The resins to which the present invention is particularly effective are those positively charged resins which contain primary and/or secondary amine groups. Such resins are described in U.S. Pat. Nos. 3,663,389; 3,947,339; and 4,116,900. In U.S. Pat. No. 3,947,339, a polyketimine derivative of a polyamine such as diethylenetriamine or triethylenetetraamine is reacted with a polyepoxide. When the reaction product is neutralized with acid and dispersed in water, free primary amine groups are generated. Also, equivalent products are formed when polyepoxide is reacted with excess polyamines such as diethylenetriamine and triethylenetetraamine and the excess polyamine vacuum stripped from the reaction mixture. Such products are described in U.S. Pat. Nos. 3,663,389 and 4,116,900.

Mixtures of the above-described ionic resins also can be used advantageously. In one embodiment of the present invention, the resin (a) comprises a polymer having cationic salt groups and is selected from a polyepoxide-based polymer having primary, secondary and/or tertiary amine groups (such as those described above) and an acrylic polymer having hydroxyl and/or amine functional groups.

Also, for purposes of the present invention, it should be understood that when the active hydrogen-containing resin comprises cationic salt groups, the boron-containing compound is present in an amount sufficient to retard the growth of microorganisms in the electrodeposition bath, but present in an amount insufficient to form the cationic salt groups. In this instance, such cationic salt groups typically are formed by solubilizing the resin with an inorganic or organic acid conventionally used in electrodepositable compositions. Suitable examples of solubilizing acids include, but are not limited to, sulfamic, acetic, lactic, and formic acids. Sulfamic and lactic acids are most commonly employed.

The active hydrogen-containing ionic electrodepositable resin described above is present in the electrodeposition bath of the invention in amounts ranging from 5 to 90 percent by weight, usually 10 to 80 percent by weight, often 10 to 70 percent by weight, and typically 10 to 30 percent by weight based on total weight of the electrodeposition bath.

As mentioned above, the resinous phase of the electrodeposition bath of the present invention further comprises (b) a curing agent adapted to react with the active hydrogen groups of the ionic electrodepositable resin (a) described immediately above. Both blocked organic polyisocyanate and aminoplast curing agents are suitable for use in the present invention, although blocked isocyanates typically are employed for cathodic electrodeposition.

Aminoplast resins, which are common curing agents for anionic electrodeposition, are the condensation products of amines or amides with aldehydes. Examples of suitable amine or amides are melamine, benzoguanamine, urea and similar compounds. Generally, the aldehyde employed is formaldehyde, although products can be made from other aldehydes such as acetaldehyde and furfural. The condensation products contain methylol groups or similar alkylol groups depending on the particular aldehyde employed. Preferably, these methylol groups are etherified by reaction with an alcohol. Various alcohols employed include monohydric alcohols containing from 1 to 4 carbon atoms such as methanol, ethanol, isopropanol, and n-butanol, with methanol being preferred. Aminoplast resins are commercially available from American Cyanamid Co. under the trademark CYMEL and from Monsanto Chemical Co. under the trademark RESIMENE.

The aminoplast curing agents typically are utilized in conjunction with the active hydrogen containing anionic electrodepositable resin in amounts ranging from about 5 percent to about 60 percent by weight, preferably from about 20 percent to about 40 percent by weight, the percentages based on the total weight of the resin solids in the electrodeposition bath.

The curing agents commonly employed in cathodic electrodeposition compositions are blocked polyisocyanates. The polyisocyanates can be fully blocked as described in U.S. Pat. No. 3,984,299 column 1 lines 1 to 68, column 2 and column 3 lines 1 to 15, or partially blocked and reacted with the polymer backbone as described in U.S. Pat. No. 3,947,338 column 2 lines 65 to 68, column 3 and column 4 lines 1 to 30, which are incorporated by reference herein. By “blocked” is meant that the isocyanate groups have been reacted with a compound such that the resultant blocked isocyanate group is stable to active hydrogens at ambient temperature but reactive with active hydrogens in the film forming polymer at elevated temperatures usually between 90° C. and 200° C.

Suitable polyisocyanates include aromatic and aliphatic polyisocyanates, including cycloaliphatic polyisocyanates and representative examples include diphenylmethane-4,4′-diisocyanate (MDI), 2,4- or 2,6-toluene diisocyanate (TDI), including mixtures thereof, p-phenylene diisocyanate, tetramethylene and hexamethylene diisocyanates, dicyclohexylmethane-4,4′-diisocyanate, isophorone diisocyanate, mixtures of phenylmethane-4,4′-diisocyanate and polymethylene polyphenylisocyanate. Higher polyisocyanates such as triisocyanates can be used. An example would include triphenylmethane-4,4′,4″-triisocyanate. Isocyanate prepolymers with polyols such as neopentyl glycol and trimethylolpropane and with polymeric polyols such as polycaprolactone diols and triols (NCO/OH equivalent ratio greater than 1) can also be used.

Also suitable are carbamate or beta-hydroxy urethane curing agents such as those described in U.S. Pat. Nos. 4,435,559 and 5,250,164. Such beta-hydroxy urethanes are formed from an isocyanate compound, for example, any of those described immediately above, a 1,2-polyol and/or a conventional blocking such as monoalcohol. Also suitable are the secondary amine blocked aliphatic and cycloaliphatic isocyanates described in U.S. Pat. Nos. 4,495,229 and 5,188,716.

The polyisocyanate curing agents are typically utilized in conjunction with the active hydrogen containing cationic electrodepositable resin in amounts ranging from ranging from 1 to 90 percent by weight, usually 1 to 80 percent by weight, often 1 to 70 percent by weight, and typically 1 to 15 percent by weight based on total weight of the electrodeposition bath.

The aqueous compositions of the present invention are in the form of an aqueous dispersion. The term “dispersion” is believed to be a two-phase transparent, translucent or opaque resinous system in which the resin is in the dispersed phase and the water is in the continuous phase. The average particle size of the resinous phase is generally less than 1.0, usually less than 0.5 microns, and typically less than 0.15 micron.

The concentration of the resinous phase in the aqueous medium is at least 1 and usually from 2 to 60 percent by weight based on total weight of the aqueous dispersion. When the compositions of the present invention are in the form of resin concentrates, they generally have a resin solids content of 20 to 60 percent by weight based on weight of the aqueous dispersion.

Electrodeposition baths of the invention typically are supplied as two components: (1) a clear resin feed, which includes, generally, the active hydrogen-containing ionic electrodepositable resin, i.e., the main film-forming polymer, the curing agent, and any additional water-dispersible, non-pigmented components; and (2) a pigment paste, which, generally, includes one or more pigments, a water-dispersible grind resin which can be the same or different from the main-film forming polymer, and, optionally, additives such as catalysts, and wetting or dispersing aids. Electrodeposition bath components (1) and (2) are dispersed in an aqueous medium which comprises water and, usually, coalescing solvents. Alternatively, the electrodeposition baths of the present invention can be supplied as one component compositions.

It should be appreciated that there are various methods by which the boron-containing compound can be incorporated into the electrodeposition bath. The boron-containing compound can be incorporated “neat”, that is, the boron-containing compound or an aqueous solution thereof can be added directly to the dispersed electrodeposition bath components (1) and (2), or if applicable, to the dispersed one-component electrodeposition composition. Alternatively, the boron-containing compound can be admixed with or dispersed in the clear resin feed (or any of the individual clear resin feed components, for example the film-forming resin or the curing agent) prior to dispersing components (1) and (2) in the aqueous medium. Further, the boron-containing compound can be admixed with or dispersed in the pigment paste, or any of the individual pigment paste components, for example, the pigment grind resin prior to dispersing components (1) and (2) in the aqueous medium. Additionally, the boron compound can be added on-line to the electrodeposition bath, to the subsequent rinse stages, and/or to the ultrafiltrate. Moreover, a boron-containing compound, for example, boric acid, can be included as a component in any of the pretreatment rinse stages (e.g., as a biocide or as an adhesion promoter) that are located upstream in the coating process, prior to the electrodeposition bath. Residual boron-containing compound can then be carried into the electrodeposition bath along with the substrate and, thereby, can be present in the bath in an amount sufficient to retard the growth of microorganisms therein.

The electrodeposition bath of the present invention has a resin solids content usually within the range of 5 to 25 percent by weight based on total weight of the electrodeposition bath.

As aforementioned, besides water, the aqueous medium may contain a coalescing solvent. Useful coalescing solvents include hydrocarbons, alcohols, esters, ethers and ketones. The preferred coalescing solvents include alcohols, polyols and ketones. Specific coalescing solvents include isopropanol, butanol, 2-ethylhexanol, isophorone, 2-methoxypentanone, ethylene and propylene glycol and glycol ethers such as monoethyl, monobutyl and monohexyl ethers of ethylene glycol. The amount of coalescing solvent is generally between about 0.01 and 25 percent and when used, preferably from about 0.05 to about 5 percent by weight based on total weight of the aqueous medium. In one particular embodiment of the present invention, glycol ether solvent is present in the electrodeposition bath in an amount of 0.5 weight percent or less, usually 0.3 weight percent or less, and typically 0.2 weight percent or less, based on total weight of the electrodeposition bath.

As discussed above, a pigment composition and, if desired, various additives such as surfactants, wetting agents or catalyst can be included in the dispersion. The pigment composition may be of the conventional type comprising pigments, for example, iron oxides, strontium chromate, lead silicate, carbon black, coal dust, titanium dioxide, talc, barium sulfate, as well as color pigments such as cadmium yellow, cadmium red, chromium yellow and the like. In one embodiment of the present invention, the electrodeposition bath is essentially free of chrome- and/or lead- containing pigments.

The pigment content of the dispersion is usually expressed as a pigment-to-resin ratio. In the practice of the invention, when pigment is employed, the pigment-to-resin ratio is usually within the range of about 0.02 to 1:1. The other additives mentioned above are usually in the dispersion in amounts ranging from 0.01 to 10 percent by weight based on weight of resin solids.

The electrodepositable coating compositions of the present invention can be applied by electrodeposition to a variety of electroconductive substrates, including metals such as untreated steel, galvanized steel, aluminum, copper, magnesium and conductive carbon coated materials. The applied voltage for electrodeposition may be varied and can be, for example, as low as 1 volt to as high as several thousand volts, but typically between 50 and 500 volts. The current density is usually between 0.5 ampere and 5 amperes per square foot and tends to decrease during electrodeposition indicating the formation of an insulating film.

After the coating has been applied by electrodeposition, it is typically thermally cured at elevated temperatures ranging from 90° to 260° C. for a period of 1 to 40 minutes.

Illustrating the invention are the following examples which, however, are not to be considered as limiting the invention to their details. All parts and percentages in the following examples as well as throughout the specification are by weight unless otherwise indicated.

### EXAMPLES

### Example 1

Part A: This example describes the preparation of a cationic electrodepositable resin which was used as a component in the electrodeposition bath of the present invention. The resin was prepared from a mixture of the following ingredients.

| | Parts by |
|---|---|
| Ingredients | weight |
| EPON ® 880¹ | 614.68 |
| Bisphenol A-ethylene oxide adduct (⅙ molar ratio) | 125 |
| Bisphenol A | 265.42 |
| Methyl isobutyl ketone | 20.51 |
| Ethyltriphenyl phosphonium iodide | 0.6 |
| Bisphenol A-ethylene oxide adduct (⅙ molar ratio) | 125 |
| Methylisobutyl ketone | 85.53 |
| Crosslinker² | 718.3 |
| Diketimine³ | 57.01 |
| N-methyl ethanolamine | 48.68 |

¹Diglycidyl ether of Bisphenol A, commercially available from Shell Oil and Chemical Co.
²Prepared by reacting 10 equivalents of polymeric MDI (Rubinate M, available from Huntsman Corporation) with 2 equivalents of 2-(2-butoxyethoxy)ethanol and 8 equivalents of 2-butoxyethanol using dibutyl tin dilaurate as a catalyst (89% solids in methyl isobutyl ketone).
³Diketimine derived from diethylenetriamine and methyl isobutyl ketone (73% solids in methyl isobutyl ketone).

The EPON® 828, the initial charge of Bisphenol A-ethylene oxide adduct, Bisphenol A, and the initial charge of methyl isobutyl ketone were added to a suitable reaction vessel and heated in a nitrogen atmosphere to a temperature of 125° C. Ethyltriphenyl phosphonium iodide then was added and the reaction mixture was allowed to exotherm to about 145° C. The reaction was held at 145° C. for 2 hours at which time the second charge of Bisphenol A-ethylene oxide adduct was added and an epoxy equivalent was obtained. The second charge of methyl isobutyl ketone, crosslinker, diketimine and N-methylethanolamine then were added in succession. The admixture was allowed to exotherm until a temperature of 100° C. was established and held at that temperature for 1 hour. The resin mixture thus obtained (1700 parts) was dispersed in aqueous medium by combining with a mixture of 32.6 parts 88% lactic acid, 7.93 parts of sulfamic acid and 1077 parts of deionized water. The dispersion was further diluted with 626 parts of deionized water and 634 parts of deionized water in successive stages then vacuum stripped to remove organic solvent. The resultant dispersion had a solids content of 41.37 percent.

Part B: This example describes the preparation of an electrodeposition bath containing the resin of Part A. The electrodeposition bath was prepared by combining the following ingredients under agitation:

| Ingredients | Parts by weight |
|---|---|
| Resin from Example 1A | 1218.2 |
| Flexibilizer¹ | 160.3 |
| Plasticizer² | 27.7 |
| Flow Control Additive³ | 72.2 |
| Pigment paste⁴ | 234.5 |
| Boric acid | 3.8 |
| Deionized water | 2083.3 |

¹Prepared as described in Example 2A in U.S. Pat. No. 6,017,431, with lactic replacing sulfamic acid.
²Reaction product of 2 moles of diethylene glycol butyl ether and 1 mole of formaldehyde, prepared as generally described in U.S. Pat. No. 4,891,111.
³Prepared as described in Examples A and B of U.S. Pat. No. 5,096,556 with sulfamic acid substituted for acetic acid, and butylcarbitol formal substituted for ethylene glycol butyl ether.
⁴Pigment and catalyst paste available from PPG Industries, Inc. as E6251.

### Comparative Example 2

This comparative example describes the preparation of an electrodeposition bath containing resin of Part A immediately above. The electrodeposition bath was prepared as described above in Example 1B, except the boric acid was replaced with deionized water.

The electrodeposition bath compositions of Example 1B and Comparative Example 2 above were ultrafiltered 20% and reconstituted with deionized water. The pH and conductivity of each bath was measured using an Accumet® Model 50 pH/conductivity meter. Phosphated steel test panels (supplied by ACT Laboratories and labeled as “C710 No Chemseal, Immersion DIW”), were electrocoated at 90° F. (32.2° C.) for two minutes at 200 volts to yield films having a dry film thickness (DFT) of approximately 1 mil (25.4 micrometers) after curing the coated test panels for 20 minutes at 350° F. (177° C.). Appearance of the test panels thus prepared was determined by visual inspection to be equivalent for Example 1B and Comparative Example 2.

TABLE 1

| | | | | DFT |
|---|---|---|---|---|
| | pH | Conductivity | Volts | (mils) |
| Example 1B | 6.12 | 1670 | 200 | 0.92 |
| Comparative Example 2B | 6.09 | 1680 | 200 | 1.02 |

Bacteria resistance of the above-described electrodeposition bath compositions and the enumeration of the bacteria are described as follows:

Bacteria Resistance Testing:

Infected ultrafiltrate from an online electrodeposition bath tank (containing E6100H, commercially available from PPG Industries, Inc.) having a bacterial count of 4.5×10⁶ cfu/mL was used to inoculate filter-sterilized ultrafiltrate (“permeate”) collected from the same line. The infected ultrafiltrate was diluted by a factor of 10⁴ into the sterile permeate. The infected permeate was added to reactors. The various acids listed below in the following Table 2 were prepared as 1 % solutions in water and tested at 2000, 1000, 500, 200, 100, and 50 ppm of active material. Reactors were rolled for 10 days at 32° C. and were plated for bacteria counts at 3, 7, and 10 days.

Enumeration of Bacteria:

The enumeration of anerobic heterotrophs in the reactors utilized the standard spread plate method as described in Standard Methods for the Examination of Water and Wastewater, 1992, section 9215C. Bacterial counts were examined using R2A agar plates. 100 μL of a suitable dilution of sample was pipetted onto the surface of the media and spread with an alcohol and flame sterilized glass rod. The plates were inverted and incubated for 3 days at 32° C. with periodic examination. Colony forming units were counted on a New Brunswick Bactronic Colony Counter. A target of 30 to 300 cfu per plate was considered a valid count.

TABLE 2

| | | Bacteria Count | | |
|---|---|---|---|---|
| Acid | Concentration | 3 day | 7 day | 10 day |
| Boric Acid | 2000 | <4.0 × 10¹ | <4.0 × 10¹ | <4.0 × 10¹ |
| | (355 ppm B) | | | |
| | 1000 | <4.0 × 10¹ | <4.0 × 10¹ | <4.0 × 10¹ |
| | (177.5 ppm B) | | | |
| | 500 | <4.0 × 10¹ | <4.0 × 10¹ | <4.0 × 10¹ |
| | (89 ppm B) | | | |
| | 200 | 3.6 × 10² | 2.8 × 10³ | 2.0 × 10⁶ |
| | (35.5 ppm B) | | | |
| | 100 | 3.0 × 10² | 5.6 × 10⁵ | 1.4 × 10⁷ |
| | (17.8 ppm B) | | | |
| | 50 | 8.8 × 10² | 4.6 × 10⁶ | 4.0 × 10³ |
| | (8.9 ppm B) | | | |
| Lactic Acid | 2000 | 2.1 × 10⁶ | 4.8 × 10⁷ | 1.1 × 10⁷ |
| | 1000 | 9.6 × 10⁶ | 3.2 × 10⁷ | <4.0 × 10⁵ |
| | 500 | 1.6 × 10⁷ | 4.0 × 10⁷ | 2.8 × 10⁶ |
| | 200 | 1.5 × 10⁶ | 1.6 × 10⁷ | 1.2 × 10⁷ |
| | 100 | 8.8 × 10⁵ | 4.4 × 10⁷ | 2.4 × 10⁶ |
| | 50 | 9.2 × 10⁵ | 4.4 × 10⁷ | 3.8 × 10⁷ |
| No Addition | NA | 3.6 × 10⁵ | 2.6 × 10⁷ | <4.0 × 10⁵ |

Note the “<” indicates absence of colonies at lowest dilution plated.

The data presented in Table 2 above illustrate that the electrodepositable compositions of the present invention which contain boric acid, retard the growth of bacteria particularly at levels of 500 ppm or greater, as compared to lactic acid in an analogous electrodepositable composition.

It will be appreciated by those skilled in the art that changes could be made to the embodiments described above without departing from the broad inventive concept thereof. It is understood, therefore, that this invention is not limited to the particular embodiments disclosed, but it is intended to cover modifications which are within the spirit and scope of the invention, as defined by the appended claims.

Therefore, we claim:

1. In an aqueous electrodeposition bath having a reduced volatile organic content, said bath comprising an aqueous electrocoating composition comprising a resinous phase dispersed in an aqueous medium, said resinous phase comprising:
(a) an active hydrogen group-containing electrodepositable resin having ionic salt groups; and
(b) a curing agent having functional groups reactive with the active hydrogen-containing groups of the resin (a),
the improvement comprising the inclusion of a boron-containing compound selected from at least one of boric acid, boric acid equivalents, and mixtures thereof in the bath in an amount sufficient to retard the growth of microorganisms in the electrodeposition bath.

2. The electrodeposition bath of claim 1, wherein the resin (a) comprises cationic salt groups and wherein the boron-containing compound is present in an amount sufficient to retard the growth of microorganisms in the electrodeposition bath composition, but present in an amount insufficient to form said cationic salt groups.

3. The electrodeposition bath of claim 1, wherein the resin (a) comprises anionic salt groups.

4. The electrodeposition bath of claim 1, wherein the volatile organic content of the bath is 0.5 pounds per gallon or less.

5. The electrodeposition bath of claim 1, wherein the volatile organic content of the bath is 0.3 pounds per gallon or less.

6. The electrodeposition bath of claim 4 further comprising a glycol ether solvent, present in ah amount of 0.3 weight percent or less based on total weight of the electrodeposition bath.

7. The electrodeposition bath of claim 1, wherein the pH of the bath is 7.0 or less.

8. The electrodeposition bath of claim 1, wherein the conductivity of the bath ranges from 500 to 3000 microsiemens.

9. The electrodeposition bath of claim 1, wherein the bath is free of lead compounds.

10. The electrodeposition bath of claim 1, wherein said boron-containing compound comprises boric acid, boric acid esters, boron oxide, and mixtures thereof.

11. The electrodeposition bath of claim 10, wherein said boron-containing compound comprises boric acid.

12. The electrodeposition bath of claim 1, wherein said boron-containing compound is present in the electrodeposition bath in an amount sufficient to provide an amount of boron ranging from 50 to 5,000 parts per million, based on total weight of the electrodeposition bath.

13. The electrodeposition bath of claim 1, wherein said boron-containing compound is present in the electrodeposition bath n in an amount sufficient to provide an amount of boron ranging from 100 to 2,000 parts per million, based on total weight of the electrodeposition bath.

14. The electrodeposition bath of claim 1, wherein the resin (a) is selected from polyepoxide-based polymers, acrylic polymers and mixtures thereof.

15. The electrodeposition bath of claim 14, wherein the resin (a) comprises a polymer selected from at least one of
a polyepoxide-based polymer having primary, secondary and/or tertiary amine functional groups, and
an acrylic polymer having hydroxyl and/or amine functional groups.

16. The electrodeposition bath of claim 1, wherein the resin (a) comprises the reaction product of an epoxide group-containing polymer and a primary or secondary amine.

17. The electrodeposition bath of claim 1, wherein the resin (a) comprises the reaction product an epoxide group-containing polymer and a secondary amine which contains ketimine groups.

18. The electrodeposition bath of claim 1, wherein the resin (a) comprises an acrylic polymer having onium salt groups.

19. The electrodeposition bath of claim 18, wherein the onium salt comprises a ternary sulfonium salt group.

20. The electrodeposition bath of claim 18, wherein the onium salt comprises a quaternary phosphonium salt group.

21. The electrodeposition bath of claim 1, wherein the resin (a) is present in the bath composition in an amount ranging from 60 to 90 weight percent based on weight of total resin solids present in the bath composition.

22. The electrodeposition bath of claim 1, wherein the curing agent (b) is selected from at least one of blocked isocyanates, aminoplast resins, and mixtures thereof.

23. The electrodeposition bath of claim 2, wherein the curing agent (b) comprises at least one blocked isocyanate.

24. The electrodeposition bath of claim 3, wherein the curing agent (b) comprises at least one aminoplast resin.

25. The electrodeposition bath of claim 1, wherein the curing agent (b) is present in an amount ranging from 10 to 40 weight percent based on weight of total resin solids present in the bath.

26. In an electrodeposition bath having a reduced volatile organic content, said electrodeposition bath comprising an aqueous electrocoating composition comprising a resinous phase dispersed in an aqueous medium, said resinous phase comprising:
(a) an active hydrogen group-containing electrodepositable resin having cationic salt groups; and
(b) a blocked isocyanate curing agent having functional groups reactive with the active hydrogen-containing groups of the resin (a),
the improvement comprising the inclusion of a boron-containing compound selected from at least one of boric acid, boric acid equivalents, and mixtures thereof in the electrodeposition bath,
wherein said boron-containing compound is present in an amount sufficient to retard the growth of microorganisms in the electrodeposition bath, but present in an amount insufficient to form the cationic salt groups.

27. The electrodeposition bath of claim 24, wherein the resin (a) comprises a polymer selected from at least one of
a polyepoxide-based polymer having primary, secondary and/or tertiary amine functional groups, and
an acrylic polymer having hydroxyl and/or amine functional groups.

28. The electrodeposition bath of claim 26, wherein the pH of the electrodeposition bath is 7.0 or less.

29. In a method of electrocoating a conductive substrate serving as a charged electrode in an electrical circuit comprising said electrode and an oppositely charged counter electrode, said electrodes being immersed in an aqueous electrodeposition bath comprising an aqueous electrocoating composition,
said method comprising passing electric current between said electrodes to cause deposition of the electrocoating composition on the substrate as a substantially continuous film,
the aqueous electrocoating composition comprising a resinous phase dispersed in an aqueous medium, said resinous phase comprising:
(a) an active hydrogen group-containing ionic electrodepositable resin, and
(b) a curing agent having functional groups reactive with the active hydrogen groups of (a),
the improvement comprising the inclusion of a boron-containing compound selected from at least one of boric acid, boric acid equivalents, and mixtures thereof in the electrodeposition bath in an amount sufficient to retard the growth of microorganisms in the electrodeposition bath.

30. The method of claim 29, wherein the resin (a) comprises cationic salt groups and wherein the boron-containing compound is present in an amount sufficient to retard the growth of microorganisms in the electrodeposition bath, but present in an amount insufficient to form the cationic salt groups.

31. The method of claim 29, wherein the resin (a) comprises anionic salt groups.

32. The method of claim 29, wherein said volatile organic content of the bath is 0.5 pounds per gallon or less.

33. The method of claim 29, wherein said volatile organic content of the bath is 0.3 pounds per gallon or less.

34. The method of claim 33, in which the electrodeposition bath comprises glycol ether solvent present in an amount of 0.3 weight percent or less based on total weight of the electrodeposition.

35. The method of claim 29, wherein the pH of the electrodeposition bath is 7.0 or less.

36. The method of claim 29, wherein the conductivity of the electrodeposition bath ranges from 500 to 3000 microsiemens.

37. The method of claim 29, wherein the electrodeposition bath is free of lead compounds.

38. The method of claim 29, wherein said boron-containing compound comprises boric acid, boric acid esters, boron oxides, and mixtures thereof.

39. The method of claim 38, wherein said boron-containing compound comprises boric acid.

40. The method of claim 29, wherein said boron-containing compound is present in the electrodeposition bath in an amount sufficient to provide an amount of boron ranging from 50 to 5000 parts per million, based on total weight of the electrodeposition bath.

41. The method of claim 29, wherein said boron-containing compound is present in the electrodeposition bath in an amount sufficient to provide an amount of boron ranging from 100 to 2000 parts per million, based on total weight of the electrodeposition bath.

42. The method of claim 29, wherein the resin (a) is selected from polyepoxide-based polymers, acrylic polymers and mixtures thereof.

43. The method of claim 40, wherein the resin (a) comprises a polymer selected from at least one of
a polyepoxide-based polymer having primary, secondary and/or tertiary amine functional groups, and
an acrylic polymer having hydroxyl and/or amine functional groups.

44. The method of claim 29, wherein the resin (a) comprises the reaction product of an epoxide group-containing polymer and a primary or secondary amine.

45. The method of claim 29, wherein the resin (a) comprises the reaction product of a polyepoxide polymer and a secondary amine which contains ketimine groups.

46. The method of claim 29, wherein the resin (a) comprises an acrylic polymer having onium salt groups.

47. The method of claim 44, wherein the onium salt comprises a ternary sulfonium salt group.

48. The method of claim 44, wherein the onium salt comprises a quaternary phosphonium salt group.

49. The method of claim 29, wherein the resin (a) is present in the electrodeposition bath in an amount ranging from 60 to 90 weight percent based on weight of total resin solids present in the electrodeposition bath.

50. The method of claim 29, wherein the curing agent (b) is selected from at least one of blocked isocyanates, aminoplast resins, and mixtures thereof.

51. The method of claim 30, wherein the curing agent (b) comprises at least one blocked isocyanate.

52. The method of claim 31, wherein the curing agent (b) comprises at least one aminoplast resin.

53. The method of claim 29, wherein the curing agent (b) is present in an amount ranging from 10 to 40 weight percent based on weight of total resin solids present in the electrodeposition bath.

54. In a method of electrocoating a conductive substrate serving as a cathode in an electrical circuit comprising said cathode and an anode, said cathode and said anode being immersed in an aqueous electrodeposition bath comprising an aqueous electrocoating composition,
said method comprising passing electric current between said cathode and said anode to cause deposition of the electrocoating composition on the substrate as a substantially continuous film,
the aqueous electrocoating composition comprising a resinous phase dispersed in an aqueous medium, said resinous phase comprising
(a) an active hydrogen group-containing electrodepositable resin having cationic salt groups; and
(b) a blocked isocyanate curing agent having functional groups reactive with the active hydrogen-containing groups of the resin
the improvement comprising the inclusion of at least one of boric acid, boric acid equivalents, and mixtures thereof in the electrodeposition bath,
wherein said boron-containing compound is present in an amount sufficient to retard the growth of microorganisms in the electrodeposition bath, but present in an amount insufficient to form the cationic salt groups.

55. The method of claim 54, wherein the resin (a) comprises a polymer selected from at least one of
a polyepoxide-based polymer having primary, secondary and/or tertiary amine functional groups, and
an acrylic polymer having hydroxyl and/or amine functional groups.

56. The electrodeposition bath of claim 54, wherein the pH of the electrodeposition bath is 7.0 or less.

57. A substrate coated by the method of claim 29.

58. A substrate coated by the method of claim 54.
